(19) United States
(12) Patent Application Publication
(10) Pub. No.: US 2006/0293508 A1
(43) Pub. Date: Dec. 28, 2006

(54) Stabilization of triflated compounds

(76) Inventors: Michael Major, Mequon, WI (US); Robert Peterson, Germantown, WI (US); Szymon Kosinski, Menomonee Falls, WI (US)

Correspondence Address: BAKER & BOTTS, 30 ROCKEFELLER PLAZA, 44TH FLOOR, NEW YORK, NY, 10112-4498, US

(21) Appl. No.: 11/449,529

(22) Filed: Jun. 8, 2006

Related U.S. Application Data
(60) Provisional application No. 60/689,131, filed on Jun. 8, 2005.

(51) Int. Cl. C07H 5/04; C07H 5/06; C07H 17/02

(52) U.S. Cl. 536/17.4; 536/118; 536/18.7

(57) ABSTRACT

Described are novel processes for the synthesis triflated sugars. These sugars are useful for the production of compounds, such as D-1-deoxynojirimycin (DNJ) and D-1-deoxygalactonojirimycin (DGJ). In particular, described is a multi-kilogram scale stabilization method for the synthesis of imino sugars.

### SPECIFICATION

This application claims priority from U.S. Provisional Patent Application Ser. No. 60/689,131, filed Jun. 8, 2005, the disclosure of which is incorporated herein by reference in its entirety.

### BACKGROUND

Trifluoromethanesulfonyl, or triflate, is a well known protecting group for hydroxyl groups. Hydroxy group, once protected with triflate, becomes a very reactive leaving group. This feature is widely used to perform nucleophilic substitution for synthetic purposes with use of alcohols. In carbohydrate chemistry the use of triflates is especially common. The triflate-protected hydroxyl group can be replaced with any nucleophile with a complete reversal of configuration in a nucleophilic substitution reaction occurring by the SN2 mechanism. Triflate also affects a mild oxidation of primary and secondary alcohols, including both unsaturated and natural alcohols; the triflated alcohols can be oxidized to the corresponding carbonyl compounds, and the leaving group is then cleaved to remove the triflate.

However, the triflated compounds are sensitive to moisture. In slow reactions, the intermediates tend to decompose and thereby cause a reduction in reaction yield. Triflate compounds can undergo elimination to unsaturated double bond, the side-product of this process being triflic acid, which, being very strong acid, can cause further accelerated decomposition. These problems become significant when scaling up reactions to multi-kilogram scale synthesis, since the large scale reaction will take much longer than the milligram or gram scale counterpart. This increase in time is due, at least in part, to the increase in time required for solvent evaporation, transfer of product to and from the reaction vessel, and the longer heating and cooling times required to reach the desired temperature. Therefore, a need exists for a means of stabilizing the triflated sugar intermediates.

The triflate itself can be stabilized. A combination of 1-benzenesulfinyl piperidine (BSP) and trifluoromethanesulfonic anhydride was found to form a metal-free thiophile that can activate thioglycosides, through glycosyl triflates in dichloromethane and reduce problems associated with triflate stability (Crich D, Smith M. J Am Chem Soc. 2001 Sep. 19; 123(37):9015-20).

To the best of the inventors' knowledge, currently there is no known simple method for the stabilizing triflate protected sugar compounds such as, for example, intermediates of D-1-deoxygalactonojirimycin (DGJ), a deoxynojirimycin analogue of D-galactose, especially for industrial scale. D-1-deoxygalactonojirimycin (DGJ) is a potent inhibitor of both α- and β-D-galactosidases. Galactosidases catalyze the hydrolysis of glycosidic linkages and are important in the metabolism of complex carbohydrates. Galactosidase inhibitors, such as DGJ, can be used in the treatment of many diseases and conditions, including diabetes (e.g., U.S. Pat. No. 4,634,765), cancer (e.g., U.S. Pat. No. 5,250,545), herpes (e.g., U.S. Pat. No. 4,957,926), HIV and Fabry Disease (Fan et al., Nat. Med. 1999 5:1, 112-5).

There are several preparations for D-1-deoxygalactonojirimycin (DGJ) published in the literature, most of which are not suitable for repetition in an industrial laboratory on a preparative scale procedure (>100 g). Some of these syntheses include a synthesis from D-glucose (Legler G, et al., Carbohydr Res. 1986 Nov. 1; 155:119-29); D-galactose (Uriel, C., Santoyo-Gonzalez, F., et al., Synlett 1999 593-595; Synthesis 1998 1787-1792); galactopyranose (Bernotas R C, et al., Carbohydr Res. 1987 Sep. 15; 167:305-11); L-tartaric acid (Aoyagi et al., J. Org. Chem. 1991, 56, 815); quebrachoitol (Chida et al., J. Chem. Soc., Chem Commun. 1994, 1247); galactofuranose (Paulsen et al., Chem. Ber. 1980, 113, 2601); benzene (Johnson et al., Tetrahedron Lett. 1995, 36, 653); arabino-hexos-5-ulose (Barili et al., tetrahedron 1997, 3407); 5-azido-1,4-lactones (Shilvock et al., Synlett, 1998, 554); doxynojirimicin (Takahashi et al, J. Carbohydr. Chem. 1998, 17, 117); acetylglucosamine (Heightman et al., Helv. Chim. Acta 1995, 78, 514); myo-inositol (Chida N, et al., Carbohydr Res. 1992 Dec. 31; 237:185-94); dioxanylpiperidene (Takahata et al., Org. Lett. 2003; 5(14); 2527-2529); and (E)-2,4-pentadienol (Martin R, et al., Org Lett. January 2000; 2(1):93-5) (Hughes A B, et al., Nat Prod Rep. April 1994; 11(2):135-62). A synthesis of N-methyl-1-deoxynojirimycin-containing oligosaccharides is described by Kiso (Bioorg Med Chem. November 1994; 2(11):1295-308). Kiso coupled protected 1-deoxynojirimycin derivative with methyl-1-thioglycosides (glycosyl donors) of D-galactose with a triflate used as the glycosyl promoter.

Fred-Robert Heiker, Alfred Matthias Schueller, Carbohydrate Research, 1986, 119-129) discloses a method for preparing DGJ in a 13 g scale, in which DGJ is isolated by stirring with ion-exchange resin and crystallized by the addition of ethanol. However, this process can not be readily adopted in an industrial scale to produce multi-kilogram quantities.

Another process for DGJ production is the procedure developed by Francisco Santoyo-Gonzalez and co-workers (Santoyo-Gonzalez, et al, Synlett 1999 593-595; Synthesis 1998 1787-1792). The strategy in this synthesis comprises: protection of the hydroxyl groups of D-galactose; triflating the resulting galactofuranoside; and converting to the altrofuranoside. The altrofuranoside is then triflated and reacted with azide to produce a 5-azido compound. This compound is then deprotected and reduced to obtain DGJ. The procedure of synthesis of DGJ as described by Santoyo-Gonzalez is more suitable for a small scale synthesis, e.g., gram quantities because its yield is very low, e.g., about 20% overall yield. One of problems with this synthesis is that the triflated furanosides are unstable and tend to decompose causing a low yield and occasionally fouling the reaction.

Therefore, there is a need for a method to stabilize triflated sugars, such as those used as intermediates of DGJ, to prevent sugars from decomposition and hydrolysis. For example, such stabilized triflated intermediates can be used to improve the overall yield of, the synthesis of DGJ from D-galactose.

### SUMMARY OF THE INVENTION

The current invention provides a method for stabilizing a triflated sugar by combining the sugar with a secondary or tertiary alkyl amine in a solvent; and removing the solvent. This provides a triflated sugar that is more stable than if the secondary or tertiary amine is not used.

In one embodiment, the triflate sugar is a tetrapivaloyl furanose or a pyranose. In another embodiment, the tertiary alkyl amine is N,N-diisopropylethyl amine, N,N,N-tributyl amine, or N,N,N-triethylamine and it provided between approximately 0.1-0.3 equivalents compared to the triflated sugar.

Another aspect of the present invention comprise a method of increasing the reaction yield of a sugar product by reacting a sugar starting material with a trifluoromethanesulfonyl reagent in a solvent to produce a triflated sugar; adding a secondary or tertiary amine to the triflated sugar; concentrating the solvent; and reducing to produce a triflated sugar. Sodium nitrite may be added to the reaction as well.

Other features, advantages and embodiments of the invention will be apparent to those skilled in the art from the following description, accompanying data and appended claims.

### BRIEF DESCRIPTION OF THE DRAWINGS

The following drawings form part of the present specification and are included to further demonstrate certain aspects of the invention. The invention may be better understood by reference to one or more of these drawings in combination with the detailed description of specific embodiments presented herein.

FIG. 1. Synthetic scheme showing the synthesis of DGJ starting from D-Galactose and having the triflated intermediates III and V.

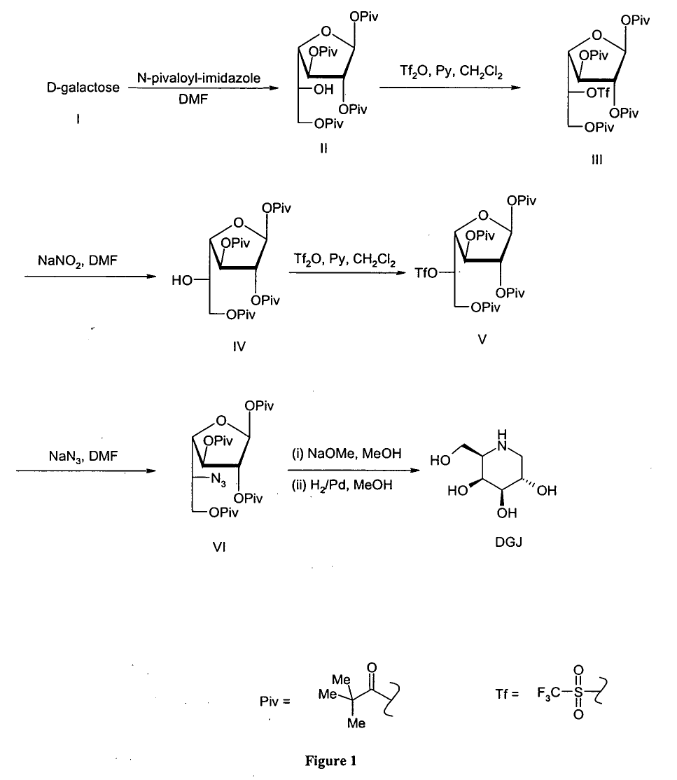

FIG. 2. Thin Layer Chromatography of Triflate III decomposition. Elution is with Hexane:Ethyl Acetate (4:1), stained with 5% sulfuric acid and heated.

FIG. 3. Pathways of triflate decomposition and triflate stabilization.

### DETAILED DESCRIPTION OF PREFERRED EMBODIMENTS

As used herein, the term “stabilize” or “stabilized” means that the stabilized compound is less likely to decompose under conditions where the compound would decompose without stabilization. Preferably, a stabilized triflate deposes less compared to the unstabilized triflate for the same period of time, e.g., a day or a week. The decomposition may be tested using a “use test” in which the stabilized and unstabilized triflates are respectively reacted with a nitrite or azide and the stabilized triflate will give higher yield of the reactions can determine. In a preferred embodiment, the term “stabilize” or “stabilized” would mean the decomposition of a stabilized triflate would not be detectable by a standard way of analysis, e.g., MR or TLC, within an hour, preferably, a day, even more preferably a week.

As used herein, the term “multi-kilogram” and “preparatory scale” denotes a scale of synthesis where product is produced in an amount greater than one kg, or, more preferably, even more than 10 kg is produced in a single pass.

As used herein, “reaction yield” means the number of grams of an isolated product compared to the number of grams of this product that could be obtained if the limiting starting material would be converted quantitatively to the product. “Increasing the reaction yield” means that the reaction yield is at least 10% greater using the inventive process than not using it. Preferably, the reaction yield is at least 20%, or 30%, or 40% greater. Even more preferably, the reaction yield is at least 50% or greater. Additionally, in a preferred embodiment, any reduction in reaction yield due to the decomposition of the intermediate is nominal.

The term ‘alkyl’ refers to a straight or branched C1-C20 hydrocarbon group consisting solely of carbon and hydrogen atoms, containing no unsaturation, and which is attached to the rest of the molecule by a single bond, e.g., methyl, ethyl, n-propyl, 1-methylethyl (isopropyl), n-butyl, n-pentyl, 1,1-dimethylethyl (t-butyl). The alkyls used herein are preferably C1-C8 alkyls.

The term “alkynyl” refers to a C2-C20 aliphatic hydrocarbon group containing at least one carbon-carbon double bond and which may be a straight or branched chain, e.g., ethanol, 1-progeny, 2-progeny (ally), iso-propenyl, 2-methyl-1-propenyl, 1-butenyl, 2-butenyl.

The term “cycloalkyl” denotes an unsaturated, non-aromatic mono- or multicyclic hydrocarbon ring system such as cyclopropyl, cyclobutyl, cyclopentyl, cyclohexyl. Examples of multicyclic cycloalkyl groups include perhydronapththyl, adamantyl and norbornyl groups bridged cyclic group or sprirobicyclic groups, e.g., spiro (4,4) non-2-yl.

The term “cycloalkalkyl” refers to a cycloalkyl as defined above directly attached to an alkyl group as defined above, which results in the creation of a stable structure such as cyclopropylmethyl, cyclobutylethyl, cyclopentylethyl.

The term “alkyl ether” refers to an alkyl group or cycloalkyl group as defined above having at least one oxygen incorporated into the alkyl chain, e.g., methyl ethyl ether, diethyl ether, tetrahydrofuran.

The term “alkyl amine” refers to an alkyl group or a cycloalkyl group as defined above having at least one nitrogen atom, e.g., n-butyl amine and tetrahydrooxazine.

The term “aryl” refers to aromatic radicals having in the range of about 6 to about 14 carbon atoms such as phenyl, naphthyl, tetrahydronapthyl, indanyl, biphenyl.

The term “arylalkyl” refers to an aryl group as defined above directly bonded to an alkyl group as defined above, e.g., —CH₂C₆H₅, and —C₂H₄C₆H₅.

The term “heterocyclic” refers to a stable 3- to 15-membered ring radical which consists of carbon atoms and from one to five heteroatoms selected from the group consisting of nitrogen, phosphorus, oxygen and sulfur. For purposes of this invention, the heterocyclic ring radical may be a monocyclic, bicyclic or tricyclic ring system, which may include fused, bridged or spiro ring systems, and the nitrogen, phosphorus, carbon, oxygen or sulfur atoms in the heterocyclic ring radical may be optionally oxidized to various oxidation states. In addition, the nitrogen atom may be optionally quaternized; and the ring radical may be partially or fully saturated (i.e., heteroaromatic or heteroaryl aromatic). Examples of such heterocyclic ring radicals include, but are not limited to, azetidinyl, acridinyl, benzodioxolyl, benzodioxanyl, benzofurnyl, carbazolyl, cinnolinyl, dioxolanyl, indolizinyl, naphthyridinyl, perhydroazepinyl, phenazinyl, phenothiazinyl, phenoxazinyl, phthalazinyl, pyridyl, pteridinyl, purinyl, quinazolinyl, quinoxalinyl, quinolinyl, isoquinolinyl, tetrazoyl, imidazolyl, tetrahydroisouinolyl, piperidinyl, piperazinyl, 2-oxopiperazinyl, 2-oxopiperidinyl, 2-oxopyrrolidinyl, 2-oxoazepinyl, azepinyl, pyrrolyl, 4-piperidonyl, pyrrolidinyl, pyrazinyl, pyrimidinyl, pyridazinyl, oxazolyl, oxazolinyl, oxasolidinyl, triazolyl, indanyl, isoxazolyl, isoxasolidinyl, morpholinyl, thiazolyl, thiazolinyl, thiazolidinyl, isothiazolyl, quinuclidinyl, isothiazolidinyl, indolyl, isoindolyl, indolinyl, isoindolinyl, octahydroindolyl, octahydroisoindolyl, quinolyl, isoquinolyl, decahydroisoquinolyl, benzimidazolyl, thiadiazolyl, benzopyranyl, benzothiazolyl, benzooxazolyl, furyl, tetrahydrofurtyl, tetrahydropyranyl, thienyl, benzothienyl, thiamorpholinyl, thiamorpholinyl sulfoxide thiamorpholinyl sulfonyl, dioxaphospholanyl, oxadiazolyl, chromanyl, isochromanyl.

The heterocyclic ring radical may be attached to the main structure at any heteroatom or carbon atom that results in the creation of a stable structure.

The term “heteroaryl” refers to a heterocyclic ring wherein the ring is aromatic.

The term “heteroarylalkyl” refers to heteroaryl ring radical as defined above directly bonded to alkyl group. The heteroarylalkyl radical may be attached to the main structure at any carbon atom from alkyl group that results in the creation of a stable structure.

The term “heterocyclyl” refers to a heterocyclic ring radical as defined above. The heterocyclyl ring radical may be attached to the main structure at any heteroatom or carbon atom that results in the creation of a stable structure.

The term “heterocyclylalkyl” refers to a heterocylic ring radical as defined above directly bonded to alkyl group. The heterocyclylalkyl radical may be attached to the main structure at carbon atom in the alkyl group that results in the creation of a stable structure.

The substituents in the ‘substituted alkyl’, ‘substituted alkenyl’ ‘substituted alkynyl’ ‘substituted cycloalkyl’ ‘substituted cycloalkalkyl’ ‘substituted cycloalkenyl’ ‘substituted arylalkyl’ ‘substituted aryl’ ‘substituted heterocyclic ring’, ‘substituted heteroaryl ring,’ ‘substituted heteroarylalkyl’, or ‘substituted heterocyclylalkyl ring’, may be the same or different with one or more selected from the groups hydrogen, hydroxyl, halogen, carboxyl, cyano, amino, nitro, oxo (═O), thio (═S), or optionally substituted groups selected from alkyl, alkoxy, alkenyl, alkynyl, aryl, arylalkyl, cycloalkyl, aryl, heteroaryl, heteroarylalkyl, heterocyclic ring, —COORx, —C(O)Rx, —C(S)Rx, —C(O)NRxRy, —C(O)ONRxRy, —NRxCONRyRz, —N(Rx)SORy, —N(Rx)SO2Ry, —(=N—N(Rx)Ry), —NRxC(O)ORy, —NRxRy, —NRxC(O)Ry-, —NRxC(S)Ry -NRxC(S)NRyRz, —SONRxRy-, —SO2NRxRy-, —ORx, —ORxC(O)NRyRz, —ORxC(O)ORy-, —OC(O)Rx, —OC(O)NRxRy, —RxNRyRz, —RxRyRz, —RxCF3, —RxNRyC(O)Rz, —RxORy, —RxC(O)ORy, —RxC(O)NRyRz, —RxC(O)Rx, —RxOC(O)Ry, —SRx, —SORx, —SO2Rx, —ONO2, wherein Rx, Ry and Rz in each of the above groups can be hydrogen atom, substituted or unsubstituted alkyl, haloalkyl, substituted or unsubstituted arylalkyl, substituted or unsubstituted aryl, substituted or unsubstituted cycloalkyl, substituted or unsubstituted cycloalkalkyl substituted or unsubstituted heterocyclic ring, substituted or unsubstituted heterocyclylalkyl, substituted or unsubstituted heteroaryl or substituted or unsubstituted heteroarylalkyl.

The term “halogen” refers to radicals of fluorine, chlorine, bromine and iodine.

A method to provide stable triflated sugars, such as galactofuranosides and altrofuranosides, is disclosed herein. These sugars can be made from simple and inexpensive sugars, such as D-galactose, and are useful in the production of imino sugars, such as DGJ (also described as (2R,3S,4R,5S)-2-hydroxymethyl-3,4,5-trihydroxypiperidine; 1-deoxy-galactostatin; or 1-deoxy-galactostatin), a nojirimycin derivative. The stable triflated sugars described herein allow for the multi-kilogram scale synthesis with high purity and good yields.

Sugars having a trifluoromethanesulfonyl protection group (triflated sugars) may be stabilized using the method of the current invention. Cyclic hexose sugars, including the furanoses and pyranoses, having a triflated moiety, may be stabilized using the methods described herein. The furanose and pyranose intermediates are described by the following structures A and B respectively. 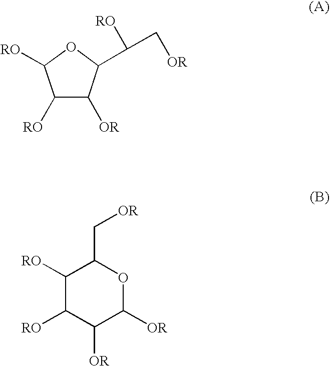
wherein at least one R is a triflate and each additional R is independently a triflate, H, substituted or unsubstituted C₁-C₁₂ alkyl, C₂-C₁₂ alkenyl, C₂-C₁₂ alkynyl, C₅-C₆ cycloalkyl, C₅-C₁₂ cycloalkenyl, C₅-C₁₂ aryl, C₄-C₁₂ heteroaryl, C₆-C₁₂ arylalkyl, C₄-C₁₂ heterocycle, C₆-C₁₂ heterocycloalkyl or C₅-C₁₂ heteroarylalkyl, OS(═O)₂R², C(═O)R², an other O-protecting group as understood in the art of carbohydrate chemistry. R² is a substituted or unsubstituted C₁-C₁₂ alkyl, C₂-C₁₂ alkenyl, C₂-C₁₂ alkynyl, C₅-C₆ cycloalkyl, C₅-C₁₂ cycloalkenyl, C₅-C₁₂ aryl, C₄-C₁₂ heteroaryl, C₆-C₁₂ arylalkyl, C₄-C₁₂ heterocycle, C₆-C_{C}₁₂ heterocycloalkyl or C₅-C₁₂ heteroarylalkyl. Some preferred R groups include: haloalkyl, polyhaloalkyl, chloroacetyl, dichloroacetyl, and trichloroacetyl. Since at least one R is a triflate protecting group, there are no free hydroxyl group present on the sugar to prevent the reaction between the triflate and the hydroxy. The triflated sugar is not triflated D-mannose.

Pentose sugars are also contemplated in the present invention. These 5-carbon sugars can be triflated and stabilized by the methods described herein. The pentose sugars may be defined by: 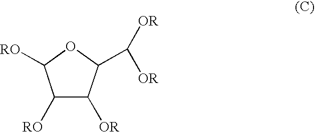
where R is defined as defined for the hexose sugars.

Heptose sugars are also contemplated in the present invention. These 7-carbon sugars can be triflated and stabilized by the methods described herein. The heptose sugars may be defined by: 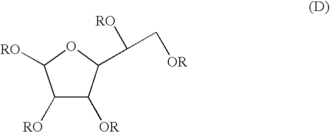
where R is defined as defined for the hexose sugars.

The triflated sugars of the present invention can be prepared by known processes. They can, for example, be triflated monosaccharides and oligosaccharides, such as mono, di-, tri-, tetra- and penta-saccharides. In one embodiment, the triflated furanose is selected from D-glucose, D-galactose, D-altrose, D-ketose, D-aldose, D-psicose, D-fructose, D-sorbose or D-tagtose. In another embodiment, the triflated pyranose is selected from D-ribose, D-arabinose, D-xylose or D-lyxose; or the triflated hexose is selected from D-allose, D-altrose, D-glucose, D-gulose, D-idose, D-galactose or D-talose, where at least one the hydroxyl group is protected with a triflate group.

Triflate-protected disaccharides and trisaccharides may also be stabilized using the methods described herein. In some embodiments, the disaccharide is trehalose, sophorose, kojibiose, laminaribiose, maltose, cellobiose, isomaltose, gentobiose, sucrose, raffinose or lactose, where at least one hydroxyl group is protected using a triflate group.

A triflated sugar is formed by reacting a sugar, such as a tetrapivaloyl furanose with any trifluoromethanesulfonylating agent, such as trifluoromethanesulfonic acid anhydride (trifluoromethanesulfonic anhydride, triflic anhydride), trifluoromethanesulfonyl chloride, N-phenyl trifluoromethanesulfonimide or the like, in the presence of a base. A preferred base for this reaction is pyridine, however, other bases, such as triethylamine, n-butylamine, N,N-dimethylaminopyridine may be used. Alkaline metal salts, such as sodium carbonate, potassium carbonate, sodium hydrogen carbonate, and potassium hydrogen carbonate, may be used as the base as long as it does not cause decomposition of the triflate when formed (e.g., the base must be relatively weak.)

The triflate sugars are useful in a variety of reactions, particularly in carbohydrate chemistry. One example of the use of a triflate sugar as a stable reaction intermediate is in the synthesis of DGJ described by Santoyo-Gonzalez, which uses D-galactose as a starting material in the synthesis of DGJ. The synthesis by Santoyo-Gonzalez can be modified by the method disclosed herein to provide a stable triflate intermediate and thereby provide a reaction scheme that allows for the synthesis of DGJ on a multi-kg scale.

In addition, the stabilized sugars are useful in reactions involving the pivoylated sugars. These sugars, which are inexpensively and simply isolated and purified by crystallization, may be used as starting materials for reactions requiring the protection of the alcohols moieties.

On a small scale (e.g., milligram quantities), the preparation of a particularly preferred triflated sugar, 5-trifluoromethanesulfonyloxy-5-deoxy-1,2,3,6-tetrapivaloyl-α-D-galactofuranose III (this sugar may also be described as 1,2,3,6-tetra-O-pivaloyl-5-O-trifluoromethanesulfonyl-α-D-galactofuranose) and its further reactions can proceed with moderate to high yields, as described by Santoyo-Gonzalez et al. In this synthesis, pivaloyl-protected sugar 1,2,3,6-tetrapivaloyl-α-D-galactofuranose A is reacted with trifluoromethanesulfonic anhydride in CH₂Cl₂ and then after work-up, immediately with sodium nitrite to yield the inverted 1,2,3,6-tetrapivaloyl-α-L-altrofuranose (IV). HPLC demonstrated the complete conversion to the inverted alcohol, due to the markedly different retentions times of D-galacto (A) and L-altro (IV) derivatives. 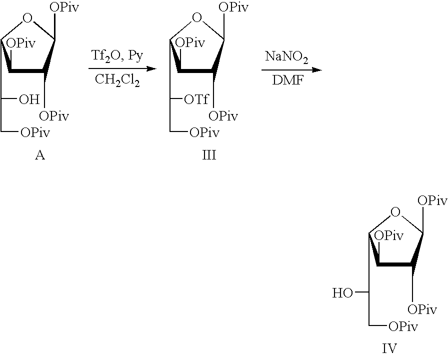
However, this inversion reaction gives only moderate yield (e.g., 30-50%) of IV, on this scale. This low yield is caused, at least in part, by the relatively unstable intermediate triflate III entering competing side elimination or hydrolysis reactions to give other products during work-up or while reacting with nitrite.

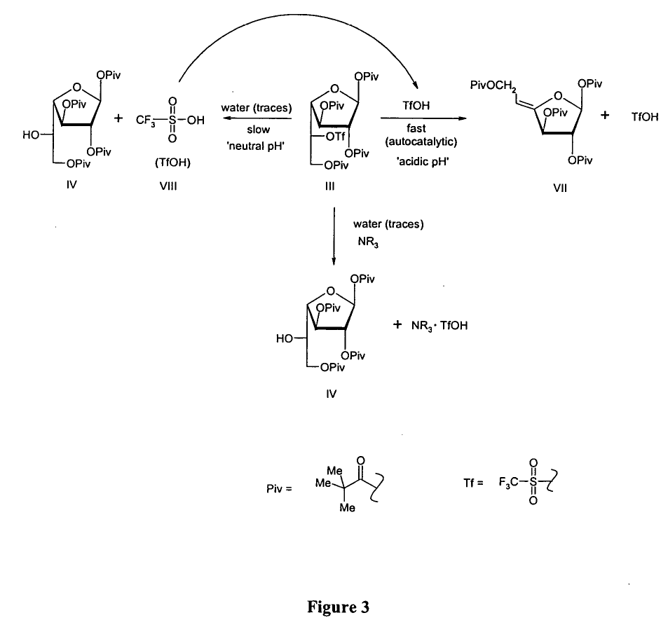

When this reaction is performed on a larger scale, the isolation of the triflate requiring the removal of solvent is even more problematic due to the corresponding larger volume of solvent to be removed. During concentration of the solvent, significant decomposition of the triflate is observed. While on the small scale triflate III can be isolated as white to off-white solids, on the kilogram scale it may be often isolated as brown solid or even liquid, which is apparent sign of decomposition. One source of this decomposition is the trace amounts of water present in the solvent (e.g., methylene chloride). One possible mechanism of this decomposition may include cleaving the triflate III to produce triflic acid and starting compound (see FIG. 3). Triflic acid also promotes further decomposition of the triflate III to form unsaturated compound VII in autocatalytic process. In some instances, as further scale-up, this process causes all of the triflate III to be completely decomposed during end stage of concentration. In addition to the triflate cleavage, the high temperature inside the flask and higher concentration of components made additional contribution to the triflate decomposition.

The decomposition of the unstable triflate can cause the pH to decrease very drastically from neutral to about 1 and then the decomposition self-accelerates. Initially, this decomposition is slow, and for small scale synthesis, stabilization may not be required. For example, the inversion reaction (galacto to altro) can be reproduced up to 500 g without stabilization with a secondary or tertiary amine. However, for larger reactions with the corresponding longer work-up times, stabilization is required.

It has now been discovered that a new procedure can be used to stabilize the unstable intermediate III as well as stabilize other triflated sugars. The addition of a secondary or tertiary amine base during the concentration step stabilizes the product, since the triflic acid formed in the initial decomposition is then quenched to form the salt IV and not allowed to catalyze any further decomposition.

Similarly, the unstable intermediate V is stabilized by combination with a secondary or tertiary amine base. This intermediate is readily converted to the corresponding azide VI.

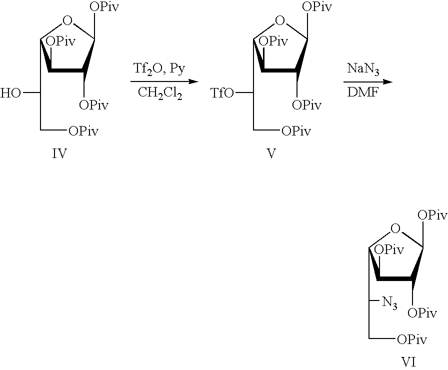

Because of the stabilization, the compound V can be obtained in a high yield. Furthermore, the amine base added does not affect the formation of the compound VI, so that a high overall yield can be achieved.

After stabilization of the triflate sugar, the triflate moiety may be removed by solvating the compound and reacting it with a compound, such as a nitrate and neutralized. The product then may be extracted with a solvent system, such as heptane/ethyl acetate, and crystallized from a solvent, such as heptane.

There are other standard procedures for work-up of triflate which are contemplated by this invention. For example, the triflate may be co-evaporated with toluene to remove pyridine. However, it is preferred to use a work-up that allows for convenient production on a large scale and minimal production of side products, such as those produced when the triflate is heated during a work-up with toluene.

The stabilized triflate prepared according to the present invention can be dried and stored for a period of time for future use without significant decomposition thereof.

Crude product, defined as compound III or V may be isolated by crystallization from solutions, such as aqueous/DMF solution. This crystallization is slow and can take up to 2 days. Once the crude product is collected, it can be dissolved in solutions, such as heptane/ethyl acetate. It can then be purified by washing, drying, concentrated, and recrystallized from, e.g., heptane, to leave the penta-pivaloylate compound in the mother liquor. This crystallization is also rather slow and may take up to 2 days. The typical yield range on this step is 30-33%. For a reaction involving D-galactose, the 1,2,3,6-tetrapivaloyl-α-D-galactofuranoside product is a white crystalline powder having high purity.

The amine base used to stabilize the triflated sugar is an organic amine that can be dissolved in the same solvent, in which the triflated sugar is prepared, and does not cause any side reaction with the triflated sugar. The organic amine is preferably a secondary or tertiary alkyl amine, more preferably a tertiary alkyl alkyl amine.

The secondary amine may includes, for example dialkyl amines having three or more carbons per alkyl chain. Preferred dialkyl amines will have 3, 4, 5, 6, 7, or 8 carbons on each alkyl chain. The tertiary amine may include trialkyl amines having one or more carbons per alkyl chain. Preferred trialkyl amines will have 3, 4, 5, 6, 7, or 8 carbons on two or three alkyl chain. The alkyl chains in both the dialkyl amines and trialkyl amines may link with each other to form a cyclic, bicyclic, or tricyclic compound.

Preferably, the base will be a hindered secondary amine or a tertiary amine. The base may be, but is not limited to Hunig's base (diisopropylethyl amine), triethyl amine, tributyl amine, diisopropylmethyl amine, diisopropylbutyl amine, diisopropylproply amine, tripropyl amine, triisopropyl amine, triisobutyl amine, tri-tert-butyl amine, diisobutylmethyl amine, diisobutylethyl amine, diisobutylpropyl amine, diisobutybutyl amine, diisopropyl amine, and di-tert-butyl amine. The organic base may also be a secondary or tertiary cyclic amine including monocyclic rings such as pyridine, morpholine, and bicyclic or tricyclic rings such as those in urotropine, or diazabicycloundecane. One particularly preferred organic base is Hunig's base.

The structure of amine base useful to stabilize the triflated compound depends on which position(s) on the sugar the triflate is located. More reactive sugars require the use of an amine base that is less reactive. For example, since the sugar C6 position is most reactive, a sugar triflated in the C6 position is not stabilized with a short (e.g., 1-3 carbon) dialkyl amine. For these compositions, a base having more alkyl carbons is preferred (e.g., diisopropyl amine).

The amine base can be used in an amount that is one molar equivalent of the triflated sugar or less, preferably 0.5 equivalents, more preferably 0.2 equivalents.

The present invention is further illustrated in the following examples, which should not be taken to limit the scope of the invention.

### EXAMPLE 1

### Preparation and Stabilization of 3-trifluoromethoxy-3-deoxy-1,2,1,8-tetrapivaloyl-α-D-galactofuranoside

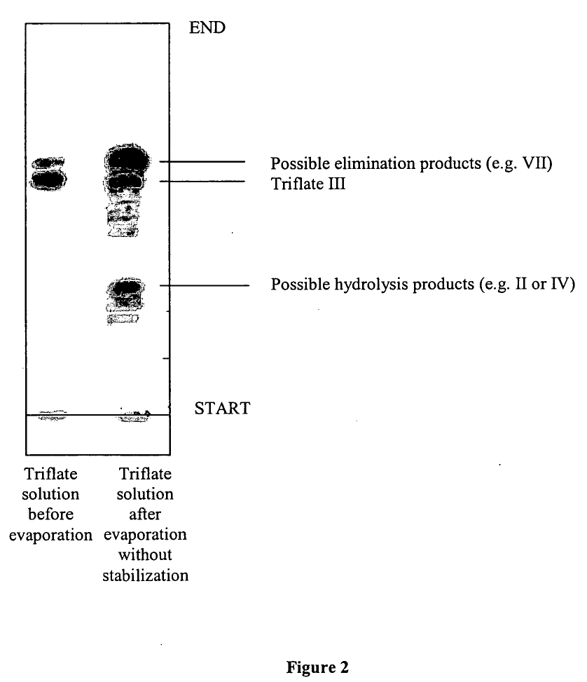

5 kg of 1,2,3,6-tetrapivaloyl-α-D-galactofuranoside was combined with 1.2 equivalents (3.3 kg) of trifluoromethanesulfonic anhydride and 5 equivalents (3.8 kg) of pyridine in 25 L of methylene chloride at 0° C. About 2 hours, the reaction mixture was with cold hydrochloric acid solution and subsequently with sodium bicarbonate solution until pH of the mixture was neutral. To methylene chloride solution of triflate was added 0.2 equivalents (230 mL) Hunig's base, and the solution was evaporated to get the titled compound. The decomposition of this compound can be seen in FIG. 2 if no base is added before evaporation.

### EXAMPLE 2

### Stabilization of Tetrapivaloyl Furanose

Following the process described in Example 1, 5 kg of a pivaloylated galactofuranoside was combined with 1.2 equivalents (3.3 kg) of trifluoromethanesulfonic anhydride and 5 equivalents (3.8 kg) of pyridine in 25 L of methylene chloride at 0° C. After about 2 hours, the reaction mixture was washed with cold hydrochloric acid solution and subsequently with sodium bicarbonate solution until pH of the mixture became neutral. To the methylene chloride solution of triflate was added 0.2 equivalents (230 mL) Hunig's base, and the solution was evaporated to get the titled compound.

### EXAMPLE 3

### Preparation and Stabilization of 3-trifluoromethoxy-3-deoxy-1,2,1,8-tetrapivaloyl-α-D-galactofuranoside

5 kg of 1,2,3,6-tetrapivaloyl-α-D-galactofuranoside 1 is combined with 1.2 equivalents (3.3 kg) of trifluoromethanesulfonic anhydride and 5 equivalents (3.8 kg) of pyridine in 25 L of methylene chloride at 0° C. After 2 hours, the reaction mixture is washed with cold hydrochloric acid solution and subsequently with sodium bicarbonate solution until pH of the mixture becomes neutral. To methylene chloride solution of triflate is added 0.2 equivalents of triethylamine, and the solution was evaporated to get the titled compound.

### EXAMPLE 4

### Stabilization of Tetrapivaloyl Furanose

Following the process described in Example 1, 5 kg of a pivaloylated galactofuranoside is combined with 1.2 equivalents (3.3 kg) of trifluoromethanesulfonic anhydride and 5 equivalents (3.8 kg) of pyridine in 25 L of methylene chloride at 0° C. After about 2 hours, the reaction mixture is washed with cold hydrochloric acid solution and subsequently with sodium bicarbonate solution until pH of the mixture becomes neutral. To the methylene chloride solution of triflate is added 0.2 equivalents of triethylamine, and the solution was evaporated to get the titled compound.

Many variations of the present invention will suggest themselves to those skilled in the art in light of the above detailed description. All such obvious variations are within the fully intended scope of the appended claims.

Those of skill in the art should, in light of the present disclosure, appreciate that many changes can be made in the specific embodiments where are disclosed herein and still obtain a like or similar result without departing from the spirit and scope of the invention.

The above mentioned patents, applications, test methods, publications are hereby incorporated by reference their entirety.

What is claimed is:

1. A method for stabilizing a triflated sugar comprising:
(a) combining a triflated sugar with an organic base in a solvent; and
(b) removing the solvent, wherein the triflated sugar is more stable than a triflated sugar not combined with the secondary or tertiary alkyl amine upon removal of solvent.

2. The method of claim 1, wherein the triflated sugar has the formula: 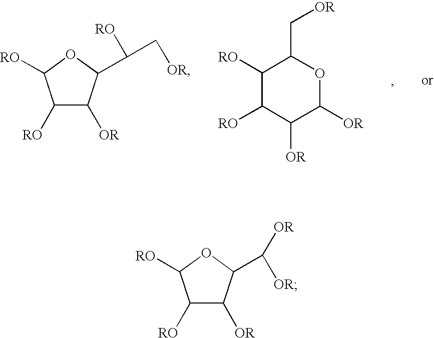
wherein at least one R is a triflate,
each additional R is independently a triflate, H, substituted or unsubstituted C₁-C₁₂ alkyl, C₂-C₁₂ alkenyl, C₂-C₁₂ alkynyl, C₅-C₆ cycloalkyl, C₅-C₁₂ cycloalkenyl, C₅-C₁₂ aryl, C₄-C₁₂ heteroaryl, C₆-C₁₂ arylalkyl, C₄-C₁₂ heterocycle, C₆-C₁₂ heterocycloalkyl, C₅-C₁₂ heteroarylalkyl, S(═O)₂R², C(═O)R², or an other O-protecting group, and
R² is a substituted or unsubstituted C₁-C₁₂ alkyl, C₂-C₁₂ alkenyl, C₂-C₁₂ alkynyl, C₅-C₆ cycloalkyl, C₅-C₁₂ cycloalkenyl, C₅-C₁₂ aryl, C₄-C₁₂ heteroaryl, C₆-C₁₂ arylalkyl, C₄-C₁₂ heterocycle, C₆-C₁₂ heterocycloalkyl or C₅-C₁₂ heteroarylalkyl.

3. The method of claim 1, wherein the triflate sugar is a tetrapivaloyl furanose.

4. The method of claim 1, wherein the organic base is a secondary or tertiary amine.

5. The method of claim 4, wherein the secondary or tertiary amine is N,N-diisopropylethyl amine, N,N,N-tributyl amine, or N,N,N-triethylamine.

6. The method of claim 1, wherein the organic base is N,N-diisopropylethyl amine.

7. The method of claim 1, wherein 0.1-0.3 equivalents of N,N-diisopropylethyl amine is used.

8. The method of claim 7, wherein the amount of N,N-diisopropylethyl amine is about 0.2 equivalents of the triflated sugar used.

9. The method of claim 2, wherein the triflate sugar is a pyranose.

10. The method of claim 2, wherein the triflate sugar is a furanose.

11. The method of claim 10, wherein the furanose is a α- D-galactofuranose.

12. The method of claim 1, wherein removing the solvent comprises evaporating the solvent to trace levels.

13. A method of increasing the reaction yield of a sugar product comprising:
(a) reacting a sugar starting material with a trifluoromethanesulfonyl reagent in a solvent to produce a triflated sugar;
(b) adding a secondary or tertiary amine to the triflated sugar; and
(c) concentrating the solvent to get a stabilized triflated.

14. The method of claim 13, wherein the amount of the secondary or tertiary amine is about 0.2 equivalents of the sugar.

15. The method of claim 13, wherein concentrating comprises evaporating the solvent to trace levels.

16. The method of claim 13, wherein the triflate sugar is a furanose.

17. The method of claim 16, further comprising: d) adding sodium nitrite to produce a furanoside, which is an isomer of the furanoside starting material.

18. The method of claim 16, wherein the sugar product is a furanoside.

19. The method of claim 13, wherein the sugar product is a pyranoside.

20. The method of claim 13, wherein the sugar product is an isomer of the sugar starting material.

21. The method of claim 13, wherein the amine is N,N-diisopropylethyl amine.

22. The method of claim 13, wherein at least 500 g of the triflated sugar is produced.

23. A stabilized triflated sugar composition comprising a secondary or tertiary alkyl amine and a triflated sugar, wherein the sugar has the formula: 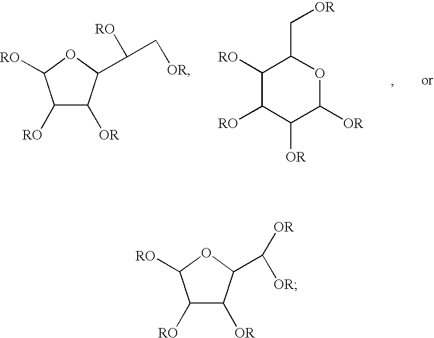
wherein at least one R is a triflate,
each additional R is independently a triflate, substituted or unsubstituted C₁-C₁₂ alkyl, C₂-C₁₂ alkenyl, C₂-C₁₂ alkynyl, C₅-C₆ cycloalkyl, C₅-C₁₂ cycloalkenyl, C₅-C₁₂ aryl, C₄-C₁₂ heteroaryl, C₆-C₁₂ arylalkyl, C₄-C₁₂ heterocycle, C₆-C₁₂ heterocycloalkyl, C₅-C₁₂ heteroarylalkyl, S(═O)₂R², C(═O)R², or an other O-protecting group, and
R² is a substituted or unsubstituted C₁-C₁₂ alkyl, C₂-C₁₂ alkenyl, C₂-C₁₂ alkynyl, C₅-C₆ cycloalkyl, C₅-C₁₂ cycloalkenyl, C₅-C₁₂ aryl, C₄-C₁₂
heteroaryl, C₆-C₁₂ arylalkyl, C₄-C₁₂ heterocycle, C₆-C₁₂ heterocycloalkyl or C₅-C₁₂ heteroarylalkyl.

24. The method of claim 23, wherein the triflate sugar is a tetrapivaloyl furanose.

25. The method of claim 23, wherein the secondary or tertiary amine is N,N-diisopropylethyl amine, N,N,N-tributyl amine, or N,N,N-triethylamine.

26. The method of claim 25, wherein the amine is N,N-diisopropylethyl amine.

27. The method of claim 26, wherein the amount of N,N-diisopropylethyl amine is 0.1-0.3 equivalents of the sugar.

28. The method of claim 27, wherein the amount is about 0.2 equivalents of the sugar.

29. The method of claim 23, wherein the triflate sugar is a pyranose.

30. The method of claim 23, wherein the triflate sugar is a furanose.

31. The method of claim 30, wherein the furanose is a α- D-galactofuranose.
